(19) United States
(12) Patent Application Publication
(10) Pub. No.: US 2019/0059971 A1
(43) Pub. Date: Feb. 28, 2019

(54) TUMOR ABLATION SYSTEM

(71) Applicant: Taiwan Earning Co. Ltd., Tainan (TW)

(72) Inventors: SHENG-CHIEH HUANG, Tainan (TW); HSIANG-SHENG WEN, Tainan (TW); TSUNG-FU YANG, Pingtung (TW); CHUNG-CHUN LIAO, Taipei (TW)

(21) Appl. No.: 15/559,101

(22) PCT Filed: May 10, 2017
(86) PCT No.: PCT/CN2017/083798

§ 371 Date: Sep. 18, 2017

Related U.S. Application Data
(60) Provisional application No. 62/335,735, filed on May 13, 2016.

(51) Int. Cl. A61B 18/08; A61B 18/10; A61B 18/02; A61B 18/06; A61N 5/10; A61L 31/14; A61L 31/16; A61L 31/02; A61L 31/10; A61L 31/04

(52) U.S. Cl. CPC A61B 18/082; A61B 18/10; A61B 18/0218; A61B 18/06; A61N 5/10; A61L 31/14; A61B 2018/00577; A61L 31/022; A61L 31/026; A61L 31/10; A61L 31/04; A61L 31/024; A61L 31/16

(57) ABSTRACT

A tumor ablation system includes a heating needle and a power supply device. The heating needle includes a rigid tubular shell. The heating needle further includes a heating element and a non-heating element; the heating element has at least one heating wire, a central cylindrical object and a distal part of the shell. The heating wire and the central cylindrical object are inside the shell. The heating wire is electrically coupled to the non-heating element, and coiled on the central cylindrical object. The distal part of the shell covers the coil formed by the heating wire on the central cylindrical object. When the heating wire is charged with electricity the heating wire generates thermal energy that is conducted to the distal part of the shell. The power supply device couples to the non-heating element, and provides electricity to the heating needle.

This application claims priority to U.S. Provisional Application No. 62/335,735, filed on May 13, 2016, which is hereby incorporated by reference in its entirety.

### FIELD OF THE INVENTION

The present disclosure relates to a tumor ablation system. The tumor ablation system can be used in the treatment of cancer or tumor. More particularly, the present disclosure provides a tumor ablation system for the treatment of solid tumors.

### BACKGROUND

Tumor or neoplasm is the abnormal growth of cells. The growth rate of neoplasm or tumor cells are often faster than normal cells. Tumor cells that are non-proliferative and responsive to treatments are benign tumors. Tumor cells that tend to spread to other locations in the body and resist treatments are malignant tumors.

Cancer or malignant tumor is the abnormal growth of malignant cells in the body. The malignant cells may originate from different types of tissues. The malignant cells derived from epithelial tissues are carcinoma, such as malignant cells originated from breast, lung, kidney thyroid, colon, prostate and stomach. The malignant cells derived from non-epithelial tissues are sarcoma, such as malignant cells originated from bone, muscle, adipose, nerve, cartilage, fibrous tissue and blood.

According to the morphology of malignant cells, cancer can be classified as solid tumor or hematological malignancy. Hematological malignancy is cancer originated from blood forming tissues, such as lymphoma, multiple myeloma and leukemia. Solid tumor is an abnormal mass of tissue formed by malignant cells, and the solid tumor can be situated in one or more locations of the body. Solid tumors often have a measurable physical mass and an observable morphology.

Treatments for solid tumors may include medication, surgical removal, chemotherapy, radiotherapy or ablation. Ablation is a physical treatment that surgically removes or destroys abnormal tissues or solid tumors. Thermal ablation refers to the destruction of tissue by elevating the temperature of target tissue. The ablated tissue can be absorbed by the body or be surgically removed from the body. Various approaches are developed for tumor ablation.

Radiofrequency ablation generates heat by electrical current. One or more electrodes or conductors are applied to the target tissue, and the electrical current heats the target tissue directly via one or more electrodes. However, radiofrequency ablation may induce heat-sink effect on the target tissue. Radiofrequency ablation also has limited therapeutic effects on target tissues with diameters larger than 3 cm. U.S. Pat. No. 8,430,870 and U.S. Pat. No. 8,491,578 disclosed a radiofrequency ablation tool, the tool includes a ferromagnetic layer covering a portion of the conductor of the tool.

Microwave ablation utilizes microwave to elevate the temperature of the target tissue. One or more probes are placed on the target tissue, and microwave energy is delivered to the target tissue to generate heat. However, microwave ablation may also induce heat-sink effect on the target tissue. When using the microwave ablation, the ablation area is difficult to be controlled, and this would result in undesired tissue damage. The hemorrhage of the target tissue during the microwave ablation would also be difficult to contain.

Laser ablation uses laser to vaporize water molecule and hemoglobin in the target tissue or cut the target tissue. However, comparing to microwave ablation or radiofrequency ablation, the ablation region for laser ablation is smaller. Another limitation of laser ablation is its' ablation effect can only reach superficial areas of the target tissue. Still another limitation of laser ablation is it requires longer therapeutic time when conducting ablation. The longer therapeutic time implies the surgery may impose higher risk for individuals receiving ablation.

Cryoablation destroys the target tissue by introducing hypothermia via one or more cryoprobes. The cryoprobes rapidly cool down the target tissue so that intracellular ice crystal may be formed to cause irreversible damage to the target tissue. Nevertheless, cryoablation generates serious heat sink effect during the process.

Electromagnetic thermotherapy utilizes electromagnetic fields to elevate the temperature of one or more probes or needles. More particularly, the electric current in a coil assembly generate an electromagnetic field which induces electric currents in one or more metal probes or needles, the temperature of the metal probes or needles is then elevated. However, the massive size of the electromagnetic field generating coil assembly is not convenient for the surgeon to use. U.S. Pat. No. 8,140,169, U.S. Pat. No. 8,361,060, U.S. Patent Publication No. 20110054455 and U.S. Pat. No. 9,095,329 disclosed electromagnetic thermotherapy devices with at least one coil assembly and one ferrous needle.

Some other thermal ablation techniques are developed to treat cancer or benign tumors. U.S. Patent Publication No. 20090216220, U.S. Pat. No. 8,251,985, U.S. Pat. No. 8,419,723, U.S. Pat. No. 8,372,065, U.S. Pat. No. 8,388,611, U.S. Patent Publication No. 20100179416 and U.S. Patent Publication No. 20110077628 disclosed thermal ablation devices using high-temperature fluid or vapor to heat the target tissue. U.S. Pat. No. 6,780,177, U.S. Pat. No. 6,872,203 and U.S. Patent Publication No. 20060167445 disclosed thermal ablation devices using electrical resistance heating.

It would be desirable to minimize the heat-sink effect when conducting thermal ablation on the target tissue. Therefore, it is an object of the present disclosure to provide a tumor ablation system that has a relatively lower heat-sink effect when conducting ablation on the target tissue.

It would also be desirable to contain the hemorrhage during the thermal ablation, because the hemorrhage poses significant risk for individual receiving thermal ablation. Therefore, it is also an object of the present disclosure to provide a tumor ablation system that minimizes the hemorrhage when conducting thermal ablation on the target tissue.

It is also an object of the present disclosure to provide a heating needle of the tumor ablation system that generates heat rapidly. The rapid elevation of temperature on the heating needle implies a shorter therapeutic time for the individual receiving thermal ablation.

It is also an object of the present disclosure to provide a tumor ablation system comprising not just thermal ablation devices, but also cryoablation or direct drug delivery devices. The tumor ablation system of the present disclosure is able to conduct multiple types of treatment on the target tissue.

### SUMMARY OF THE INVENTION

An embodiment of the present disclosure provides a tumor ablation system. The tumor ablation system comprises a heating needle and a power supply device. The heating needle is a rigid tubular structure comprising a heating element and a non-heating element, and the heating element comprises at least one heating wire and a central cylindrical object. The heating wire and the central cylindrical object is inside the heating needle. The heating wire is electrically coupled to the non-heating element, and the at least one heating wire is coiled on the central cylindrical object in some embodiments. The heating element of the heating needle generates therapeutic thermal energy when the heating wire is charged with electricity. The heating needle is electrically coupled to the power supply device, and the power supply device provides electricity to the heating needle.

Another embodiment of the present invention provides a method for treatment of tumor. The method includes the steps of identifying the location of the tumor; and thermally ablating the tumor by using the aforementioned tumor ablation system.

### BRIEF DESCRIPTION OF THE DRAWINGS

Implementations of the present technology will now be described, by way of example only, with reference to the attached figures.

FIG. 1 is a cross-sectional side view of the heating needle in accordance with one embodiment of the present disclosure.

FIG. 2 is a cross-sectional side view of another heating needle in accordance with one embodiment of the present disclosure.

FIG. 3 is a cross-sectional side view of another heating needle in accordance with one embodiment of the present disclosure.

FIG. 4 is a cross-sectional side view of another heating needle in accordance with one embodiment of the present disclosure.

FIG. 5 is a cross-sectional side view of another heating needle in accordance with one embodiment of the present disclosure.

FIG. 6 is a cross-sectional side view of another heating needle in accordance with one embodiment of the present disclosure.

FIG. 7 is a cross-sectional side view of another heating needle in accordance with one embodiment of the present disclosure.

FIG. 8A and FIG. 8B are top and side views, respectively of the holder in accordance with one embodiment of the present disclosure.

FIG. 9A, FIG. 9B, FIG. 9C, FIG. 9D and FIG. 9E illustrates the muscle tissues after having gone through thermal ablation in-vitro with different voltages by the tumor ablation system in accordance with one embodiment of the present disclosure.

FIG. 10A, FIG. 10B, FIG. 10C, FIG. 10D and FIG. 10E illustrate different stages of the muscle tissues after having gone through thermal ablation in-vitro with different ablation times by the tumor ablation system in accordance with one embodiment of the present disclosure.

FIG. 11 is a photo of an animal undergoing a thermal ablation procedure in accordance with one embodiment of the present disclosure.

FIG. 12A and FIG. 12B show changes in melanoma tumor before a thermal ablation procedure and after the procedure, according to one embodiment of the present disclosure.

### DETAILED DESCRIPTION OF THE INVENTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

The present disclosure is directed to a tumor ablation system. The tumor ablation system comprises a heating needle and a power supply device. The heating needle is a rigid tubular structure comprising a heating element and a non-heating element, and the heating element comprises at least one heating wire and a central cylindrical object. The heating wire and the central cylindrical object is inside the heating needle. The heating wire is electrically coupled to the non-heating element, and the at least one heating wire is coiled on the central cylindrical object in some embodiments. The heating element of the heating needle generates therapeutic thermal energy when the heating wire is charged with electricity. The heating needle is electrically coupled to the power supply device, and the power supply device provides electricity to the heating needle.

The present disclosure is further directed to a heating needle. The heating needle is a rigid tubular structure comprising a heating element and a non-heating element. The heating element comprises at least one heating wire and a central cylindrical object, and the at least one heating wire is coiled on the central cylindrical object, wherein the heating element generates therapeutic thermal energy when the heating wire is charged with electricity. The non-heating element is electrically coupled to the heating wire.

According to one or more exemplary embodiments, the heating needle includes a distal end and a proximal end, wherein a heating needle orifice is located at the distal end of the heating needle. The heating needle further comprises a heating needle filler. The heating needle filler can be filled in the space between the heating needle and the central cylindrical object. The heating needle filler can be removed from the space by the heating needle orifice. The heating needle filler can be an inert gas, an ultrasound contrast agent, a thermally sensitive material, a chemical ablation agent or a medication for tumor. The inert gas can be any one of helium (He), neon (Ne), argon (Ar), krypton (Kr) or xenon (Xe). The ultrasound contrast agent can be any commercially approved microbubble-based contrast agents, such as Optson™, Definity®, SonoVue® or Sonazoid™. The thermally sensitive material can be any one of metal particles, temperature-responsive polymer particles, or polymer-coated metal particles. The chemical ablation agents can be any one of ethanol, inorganic acid solutions or inorganic base solutions.

According to one or more exemplary embodiments, the central cylindrical object is a central tube. The central tube includes a distal end and a proximal end, wherein a central tube orifice is located at the distal end of the heating needle. The central tube further comprises a central tube filler. The central tube filler can be filled in the central tube. The central tube filler can be removed from the central tube by the central tube orifice. The central tube filler can be a thermally sensitive material, a chemical ablation agent or a medication for tumor. The thermally sensitive material can be any one of metal particles, temperature-responsive polymer particles, or polymer-coated metal particles. The chemical ablation agents can be any one of ethanol, inorganic acid solutions or inorganic base solutions.

According to one or more exemplary embodiments, the non-heating element comprises a plurality of insulated wires. The insulated wires electrically couple to different parts of the heating wire to form different electrical connections. The different electrical connections may be charged with electricity to generate heat on different parts on the heating wire. The heating needle may comprise 2, 3, 4, 5, 6, 7 or 8 different electrical connections.

According to one or more exemplary embodiments, the heating wire comprises a plurality of heating wires of different electrical resistance. The heating wires of different electrical resistances lead to different temperatures when charged with electricity. The heating wire may comprise 2, 3, 4, 5, 6, 7 or 8 different electrical resistances.

According to one or more exemplary embodiments, the tumor ablation system further comprises a peripheral passage. The peripheral passage spirally surrounds the heating wire. The peripheral passage includes a distal end and a proximal end, wherein a plurality of orifices is located at the distal end of the peripheral passage. The peripheral passage further comprises peripheral passage fillers, and the peripheral passage fillers is filled in the peripheral passage, wherein the peripheral passage fillers is removed from the peripheral passage by the plurality of orifices of the peripheral passage. The peripheral passage further comprises a peripheral passage filler. The peripheral passage filler can be an inert gas, a thermally sensitive material, a chemical ablation agent, a cryoablation agent or a medication for tumor. The inert gas can be any one of helium (He), neon (Ne), argon (Ar), krypton (Kr) or xenon (Xe). The thermally sensitive material can be any one of metal particles, temperature-responsive polymer particles, or polymer-coated metal particles. The chemical ablation agents can be any one of ethanol, inorganic acid solutions or inorganic base solutions. The cryoablation agent can be liquid nitrogen (N₂) or high-pressure argon (Ar).

According to one or more exemplary embodiment of the present disclosure, the tumor ablation system further comprises a connecting device. The connecting device is electrically coupled to the heating wire and power supply device. The connecting device can be one or more wires wrapped with insulators, such as fabrics or polymers.

According to one or more exemplary embodiment of the present disclosure, the tumor ablation system further comprises a measuring needle. The measuring needle measures the temperature during the thermal ablation. The measuring needle can be a thermistor or a thermocoupler.

According to one or more exemplary embodiment of the present disclosure, the tumor ablation system further comprises a syringe. The syringe is filled with inert gas, an ultrasound contrast agent, a thermally sensitive material, a chemical ablation agent, a cryoablation agent or a medication for tumor. The inert gas can be any one of helium (He), neon (Ne), argon (Ar), krypton (Kr) or xenon (Xe). The ultrasound contrast agent can be any commercially approved microbubble-based contrast agents, such as Optson™, Definity®, SonoVue® or Sonazoid™. The thermally sensitive material can be any one of metal particles, temperature-responsive polymer particles, or polymer-coated metal particles. The chemical ablation agents can be any one of ethanol, inorganic acid solutions or inorganic base solutions. The cryoablation agent can be liquid nitrogen (N₂) or high-pressure argon (Ar).

According to one or more exemplary embodiment of the present disclosure, the tumor ablation system further comprises a holder. The holder is a flat structure supporting and fixing components of the tumor ablation system. The components of the tumor system supported and fixed by the holder can be any one or more of the heating needle, the measuring needle, the connecting device and the syringe.

The present disclosure is further directed to a method for tumor treatment. The method includes: a) identify the location of the target tissue and b) thermally ablate the target tissue by using a tumor ablation system. The tumor ablation system comprises a heating needle and a power supply device. The heating needle is a rigid tubular structure having a heating element and a non-heating element, and the heating element comprises at least one heating wire and a central cylindrical object. The heating wire and the central cylindrical object is inside the heating needle. The heating wire is electrically coupled to the non-heating element, and the at least one heating wire is coiled on the central cylindrical object. The heating element of the heating needle generates thermal energy when the heating wire is charged with electricity. The heating needle is electrically coupled to the power supply device, and the power supply device provides electricity to the heating needle.

According to one or more exemplary embodiments of the present disclosure, the method for tumor treatment further comprises surgically removing the target tissue, exposing the target tissue to therapeutic radiation, exposing the target tissue to one or more chemical ablation agent or exposing the target tissue to one or more cryoablation agent after thermally ablating the target tissue. The chemical ablation agents can be any one of ethanol, inorganic acid solutions or inorganic base solutions. The cryoablation agent can be liquid nitrogen (N₂) or high-pressure argon (Ar).

The term “ablation” refers to the process that surgically removes abnormal tissues or solid tumors. The term “thermal ablation” refers to the destruction of tissue by elevating the temperature of target tissue. The term “surgeon” or “user” refer to the individual conducting ablation or thermal ablation on in-vitro animal tissues, animal bodies or human bodies. The term “distal” refers to a position on the components of the tumor ablation system or the heating needle that is away from the user. The term “proximal” refers to a position on the components of the tumor ablation system or the heating needle that is close to the user and distant to the “distal” position on the tumor ablation system or the heating needle.

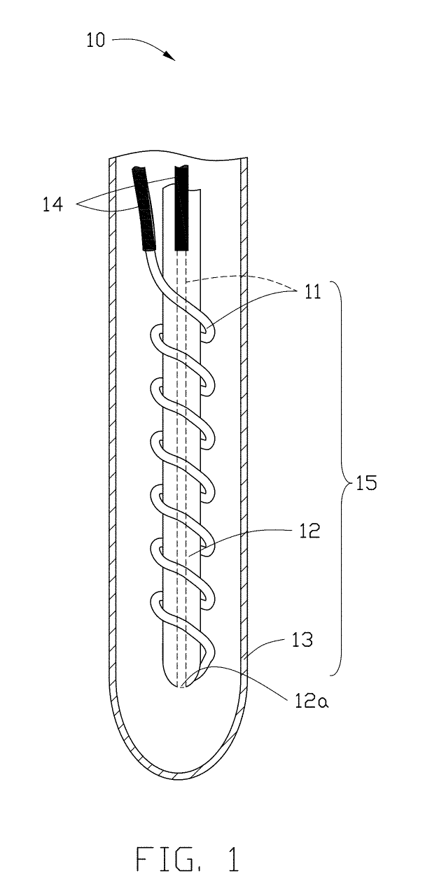

FIG. 1 is a cross-sectional side view of a heating needle 10, according to one embodiment of the present disclosure. The heating needle 10 directly contacts a target tissue and provides heat to thermally ablate the target tissue. The heating needle 10 is a rigid tubular structure and the rigid tubular structure may include a shell in some embodiments and part or all of the shell of the heating needle 10 is resistant to physical deformation under normal atmospheric pressure and temperature (e.g., room temperature). The shell of the heating needle 10 is composed of a thermal-conductive material to efficiently conduct thermal energy to the target tissue. The thermal conductive material may be ceramic, metal, carbon fiber or other materials of high thermal conductivity. In one embodiment, the shell of the heating needle 10 is composed of stainless steel. In some embodiments, the external surface of the shell is coated with a surface treatment such as, for example nanogold, nanosilver or Teflon® to form a non-stick surface. In other embodiments, polishing or plasma treatment may be applied to the external surface of the shell to form a non-stick surface.

The heating needle 10 has a diameter of about 1.8 mm to about 3 mm and a length of about 100 mm to about 200 mm in some embodiments. Preferably, the heating needle 10 has a diameter of about 2.4 mm and a length of about 150 mm.

The heating needle 10 also includes a heating element 15 and a non-heating element 14. The heating element 15 is the part of the heating needle 10 that conducts thermal energy to the target tissue when using the tumor ablation system. The heating element 15 comprises at least one heating wire 11, a central tube 12 and the distal part of the shell 13.

The heating wire 11 is composed of high resistance wire capable of generating heat when charged with electricity. The high resistance wire can be nichrome wire or tungsten wire in some embodiments, and can be wrapped with insulator to prevent short circuit. The heating wire 11 is located within the heating needle 10 and is coiled on the central tube 12. The distance between each turns of the coil formed by the heating wire 11 is no greater than 5 mm in some embodiments. The larger the distance between each turns, the longer it takes for the heating wire 11 to be elevated to a designated temperature, and the designated temperature can be the ablation temperature. Preferably, the distance between each turns of the coil formed by the heating wire 11 is about 1 mm to about 2 mm. The coil formed by the heating wire 11 has around 5 to 20 turns in some embodiments. Preferably, the coil formed by the heating wire 11 has 8 to 10 turns.

The central tube 12 is a tube with at least one central tube orifice 12a located at the distal end of the central tube 12. The central tube orifice 12a is an entrance for the heating wire 11 to enter the central tube 12. The portion of the heating wire 11 inside the central tube 12 is electrically coupled to the non-heating element 14. The central tube orifice 12a has a diameter of about 0.4 mm to about 1 mm in some embodiments. The central tube 12 is a hollow structure centrally located in the heating needle 10, and the central tube 12 can be coiled by the heating wire 11. The central tube 12 has a diameter of about 1 mm in one embodiment. The length of the central tube 12 is about 120 mm to about 100 mm shorter than the heating needle 10, therefore the length of the central tube 12 is about 50 mm to about 20 mm. The central tube 12 is composed of an insulator, such as ceramic, synthetic polymer, synthetic polymer-coated ceramic and synthetic polymer-coated metal. Preferably, the central tube 12 is a ceramic tube.

When using the tumor ablation system, the heat generated by the heating wire 11 is conducted to the target tissue during thermal ablation by the distal part of the shell 13. The distal part of the shell 13 conducts the thermal energy from the heating wire 11 charged with electricity and conducts said thermal energy to the target tissue. The distal part of the shell 13 is defined by the area of the shell covering the coil formed by the heating wire 11, and the distal part of the shell 13 becomes a heated segment of the shell when the heating wire 11 is charged with electricity. The length of the distal part of the shell 13 corresponds to the length of the coil formed by the heating wire 11. The distal part of the shell 13 has a length of about 5 cm to about 2 cm. In one embodiment, the distal part of the shell 13 has a length of about 3 cm. When using the tumor ablation system, the user may hold the part of the heating needle 10 that is proximal to the user and distant to the distal part of the shell 13 of the heating needle 10 in order to avoid heat related injury to the user.

The non-heating element 14 is electrically coupled to the heating wire 11 and is located in the heating needle 10. The non-heating element 14 is also electrically coupled to a power supply device (not shown). In order to effectively charge the heating wire 11 with electricity, the non-heating element 14 is an insulated wire of relatively lower resistance compared to the heating wire 11. The non-heating element 14 can be a conductor such as a copper wire, gold wire or silver wire and has a length of about 7 cm to about 15 cm.

The heating needle 10 is electrically coupled to a power supply device. The electricity provided to the heating needle 10 is direct current (DC), and the electricity has a voltage ranging from about 0.1V to about 10V. Preferably, the electricity provided to the heating needle 10 ranges from about 3V to about 5.5V. The ablation temperature ranges from about 70° C. to 180° C., and is positively correlated to the voltage of the electricity provided to the heating needle 10. The power supply device comprises an user interface, a transformer and an inverter in some embodiments. The power supply device may be electrically coupled to an AC or DC power source. If the power supply device is electrically coupled to an AC power source, then the transformer may convert the input AC into an output DC. The user interface of the power supply device controls the time and temperature of the heating needle for the thermal ablation. The surgeon may adjust the ablation time and temperature of the heating needle 10 for the thermal ablation via the user interface of the power supply device. The ablation time and temperature for the thermal ablation are in accordance with pathological classification and the size of the tumor.

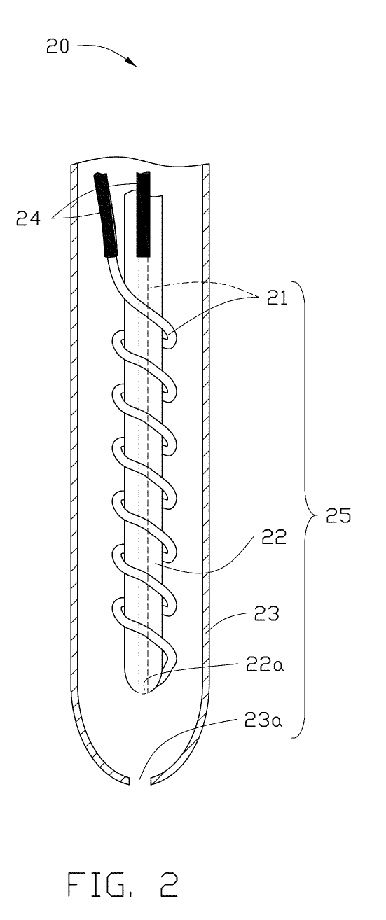

FIG. 2 is a cross-sectional side view of a heating needle, according to one embodiment of the present disclosure. The heating needle 20 directly contacts a target tissue and provides heat to thermally ablate the target tissue. The heating needle 20 is a rigid tubular structure and the rigid tubular structure may include a shell in some embodiments and part or all of the shell of the heating needle 20 is resistant to deformation under normal atmospheric pressure and temperature (e.g., room temperature). The shell of the heating needle 20 is composed of a thermal-conductive material to efficiently conduct thermal energy to the target tissue. The thermal conductive material may be ceramic, metal, carbon fiber or other materials of high thermal conductivity. In one embodiment, the shell of the heating needle 20 is composed of stainless steel. In some embodiments, the external surface of the shell is coated with a surface treatment such as, for example nanogold, nanosilver or Teflon® to form a non-stick surface. In other embodiments, polishing or plasma treatment may be applied to the external surface of the shell to form a non-stick surface.

The heating needle 20 has a diameter of about 1.8 mm to about 3 mm and a length of about 100 m to about 200 mm in some embodiments. Preferably, the heating needle 20 has a diameter of about 2.4 mm and a length of about 150 mm.

The heating needle 20 also includes a heating element 25 and a non-heating element 24. The heating element 25 is the part of the heating needle 10 that conducts thermal energy to the target tissue when using the tumor ablation system. The heating element 25 comprises at least one heating wire 21, a central tube 22 and the distal part of the shell 23.

The heating wire 21 is composed of high resistance wire capable of generating heat when charged with electricity. The high resistance wire can be nichrome wire or tungsten wire in some embodiments, and can be wrapped with insulator to prevent short circuit. The heating wire 21 is located within the heating needle 20 and is coiled on the central tube 22. The distance between each turns of the coil formed by the heating wire 21 is no greater than 5 mm in some embodiments. The larger the distance between each turns, the longer it takes for the heating wire 21 to be elevated to a designated temperature, and the designated temperature can be the ablation temperature. Preferably, the distance between each turns of the coil formed by the heating wire 11 is about 1 mm to about 2 mm. The coil formed by the heating wire 21 has around 5 to 20 turns in some embodiments. Preferably, the coil formed by the heating wire 21 has 8 to 10 turns.

The central tube 22 is a tube with at least one central tube orifice 22a located at the distal end of the central tube 22. The central tube orifice 22a is an entrance for the heating wire 21 to enter the central tube 22. The portion of the heating wire 21 inside the central tube 22 is electrically coupled to the non-heating element 24. The central tube orifice 22a has a diameter of about 0.5 mm to about 1 mm in some embodiments. The central tube 22 is a hollow structure centrally located in the heating needle 20, and the central tube 22 can be coiled by the heating wire 21. The central tube 22 has a diameter of about 1 mm in one embodiment. The length of the central tube 22 is about 120 mm to about 100 mm shorter than the heating needle 20, therefore the length of the central tube 22 is about 50 mm to about 20 mm. The central tube 22 is composed of an insulator, synthetic polymer, synthetic polymer-coated ceramic and synthetic polymer-coated metal. Preferably, the central tube 22 is a ceramic tube.

A central tube filler may be filled in the central tube 22. The central tube filler can be a thermally sensitive material, a chemical ablation agent or a medication for tumor. The thermally sensitive material can be any one of metal particles, temperature-responsive polymer particles, or polymer-coated metal particles. The chemical ablation agents can be any one of ethanol, inorganic acid solutions or inorganic base solutions. The central tube filler is removed from the central tube 22 by the central tube orifice 22a using a syringe or pump mechanism.

When using the tumor ablation system, the heat generated by the heating wire 21 is conducted to the target tissue during thermal ablation by the distal part of the shell 23. The distal part of the shell 23 of the heating needle 20 conducts the thermal energy from the heating wire 21 charged with electricity, and conducts said thermal energy to the target tissue. The distal part of the shell 23 is defined by the area of the shell covering the coil formed by the heating wire 21, and the distal part 23 becomes a heated segment of the shell when the heating wire 21 is charged with electricity. The length of the distal part of the shell 23 corresponds to the length of the coil formed by the heating wire 21. The distal part of the shell 23 has a length of about 5 cm to about 2 cm. In one embodiment, the distal part of the shell 23 has a length of about 3 cm. When using the tumor ablation system, the user may hold the part of the heating needle 20 that is proximal to the user and distant to the distal part of the shell 23 of the heating needle 20 in order to avoid heat related injury to the user. The distal part of the shell 23 further comprises a heating needle orifice 23a. The heating needle orifice 23a is located at the distal end of the distal part of the shell 23. The heating needle orifice 23a has a diameter of about 0.5 mm to 1 mm. When the central tube filler reaches the space between the heating wire 21 and the shell of the heating needle 20, the central tube filler can be removed from the heating needle 20 by the heating needle orifice 23a. The central tube filler can be removed from the heating needle 20 before, during or after the thermal ablation.

The non-heating element 24 is electrically coupled to the heating wire 21 and is located in the heating needle 20. The non-heating element 24 is also electrically coupled to a power supply device (not shown). In order to effectively charge the heating wire 21 with electricity, the non-heating element 24 is an insulated wire of relatively lower resistance compared to the heating wire 21. The non-heating element 24 can be a conductor such as a copper wire, gold wire or silver wire and has a length of about 7 cm to about 15 cm.

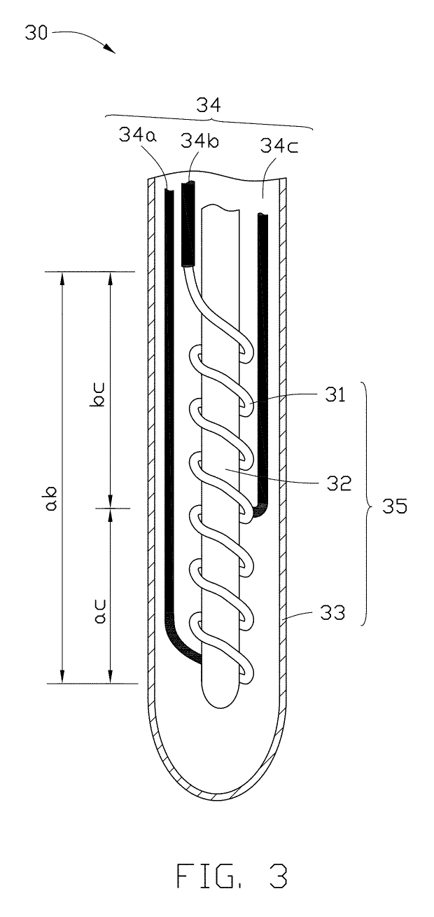

FIG. 3 is a cross-sectional side view of a heating needle 30, according to one embodiment of the present disclosure. The heating needle 30 directly contacts a target tissue and provides heat to thermally ablate the target tissue. The heating needle 30 is a rigid tubular structure, and the rigid tubular structure may include a shell in some embodiments and part or all of the shell of the heating needle 30 is resistant to physical deformation under normal atmospheric pressure and temperature (e.g. room temperature). The shell of the heating needle 30 is composed of a thermal-conductive material to efficiently conduct thermal energy to the target tissue. The thermal conductive material may be ceramic, metal, carbon fiber or other materials of high thermal conductivity. Preferably, the shell of the heating needle 30 is composed of stainless steel. In one embodiment, the external surface of the shell is coated with a surface treatment such as, for example nanogold, nanosilver or Teflon® to form a non-stick surface. In other embodiments, polishing or plasma treatment may be applied to the external surface of the shell to form a non-stick surface.

The heating needle 30 has a diameter of about 1.8 mm to about 3 mm and a length of about 100 mm to about 200 mm in some embodiments. Preferably, the heating needle 10 has a diameter of about 2.4 mm and a length of about 150 mm.

The heating needle 30 also includes a heating element 35 and a non-heating element 34. The heating element 35 is the part of the heating needle 30 that conducts thermal energy to the target tissue when using the tumor ablation system. The heating element 35 comprises at least one heating wire 31, a central tube 32 and the distal part of the shell 33.

The heating wire 31 is composed of high resistance wire capable of generating heat when charged with electricity. The high resistance wire can be nichrome wire or tungsten wire in some embodiments, and can be wrapped with insulator to prevent short circuit. The heating wire 31 is located within the heating needle 30 and is coiled on the central tube 32. The distance between each turns of the coil formed by the heating wire 31 is no greater than 5 mm in some embodiments. The larger the distance between each turns, the longer it takes for the heating wire 31 to be elevated to a designated temperature, and the designated temperature can be the ablation temperature. Preferably, the distance between each turns of the coil formed by the heating wire 31 is about 1 mm to about 2 mm. The coil formed by the heating wire 31 has around 5 to 20 turns in some embodiments. Preferably, the coil forming by the heating wire 31 has 8 to 10 turns.

The central tube 32 is a hollow structure centrally located in the heating needle 30, and the central tube 32 can be coiled by the heating wire 31. The central tube 32 has a diameter of 1 mm in one embodiment. The length of the central tube 32 is about 120 mm to about 100 mm shorter than the heating needle 30, therefore the length of the central tube 32 is about 50 mm to about 20 mm. The central tube 32 is composed of an insulator, synthetic polymer, synthetic polymer-coated ceramic and synthetic polymer-coated metal. Preferably, the central tube 32 is a ceramic tube.

The non-heating element 34 is electrically coupled to the heating wire 31 and is located in the heating needle 30. The non-heating element 34 is also electrically coupled to the power device (not shown). In order to effectively charge the heating wire 31 with electricity, the non-heating element 34 is an insulated wire of relatively lower resistance compared to the heating wire 31. The non-heating element 34 can be a conductor such as a copper wire, gold wire or silver wire and has a length of about 7 cm to about 15 cm.

The non-heating element 34 further comprises an insulated wire 34a, an insulated wire 34b and an insulated wire 34c. Each of the insulated wire 34a, the insulated wire 34b and the insulated wire 34c is electrically coupled to different parts of the heating wire 31. Multiple circuit arrangements can be formed when different parts of the heating wire 31 are connected with the insulated wire 34a, the insulated wire 34b and the insulated wire 34c. An electrical connection ab is a circuit formed by connecting the insulated wire 34a, the heating wire 31, the insulated wire 34b and the power supply device. An electrical connection be is a circuit formed by connecting the insulated wire 34b, the heating wire 31, the insulated wire 34c and the power supply device. An electrical connection ac is a circuit formed by connecting the insulated wire 34a, the heating wire 31, the insulated wire 34c and the power supply device. The electrical connection ab, the electrical connection be and the electrical connection ac charge different parts of the heating wire 31, therefore different parts of the heating wire 31 would be heated. The different heated parts of the heating wire 31 would conduct heat to different segments of the shell of the heating needle 30. A segment of the shell covering the electrical connection ab is heated when the circuit of the electrical connection ab is formed. A segment of the shell covering the electrical connection be is heated when the circuit of the electrical connection be is formed. A segment of the shell covering the electrical connection ab is heated when the circuit of the electrical connection ab is formed. When using the tumor ablation system, the user may select the electrical connection ab, the electrical connection bc or the electrical connection ac to generate thermal energy for different heated segments of the shell of the heating needle 30.

When using the tumor ablation system, the heat generated by the heating wire 31 is conducted to the target tissue during thermal ablation by the distal part of the shell 33. The distal part of the shell 33 conducts the thermal energy from the heating wire 31 charged with electricity and conduct said thermal energy to the target tissue. The distal part of the shell 33 is defined by the area of the shell covering the coil formed by the heating wire 31, and the distal part of the shell 33 becomes single or multiple heated segment of the shell when the electrical connection ab, the electrical connection bc or the electrical connection ac is formed. The length of the distal part of the shell 33 corresponds to the length of the coil formed by the heating wire 31. The distal part of the shell 33 has a length of about 5 cm to about 2 cm. In one embodiment, the distal part of the shell 33 has a length of 3 cm. When using the tumor ablation system, the user may hold the part of the heating needle 30 that is proximal to the user and distant to the distal part of the shell 33 of the heating needle 30 in order to avoid heat related injury to the user.

The heating needle 30 is electrically coupled to a power supply device. The electricity provided to the heating needle 30 is direct current (DC), and the electricity has a voltage ranging from about 0.1V to about 10V. Preferably, the electricity provided to the heating needle 30 ranges from about 3V to about 5.5V. The ablation temperature ranges from about 70° C. to 180° C., and is positively correlated to the voltage of the electricity provided to the heating needle 30. The power supply device comprises an user interface, a transformer and an inverter in some embodiments. The power supply device may be electrically coupled to an AC or DC power source. If the power supply device is electrically coupled to an AC power source, then the transformer may convert the input AC into an output DC. The user interface of the power supply device controls the time and temperature of the heating needle for the thermal ablation. The surgeon may adjust the ablation time and temperature of the heating needle 30 for the thermal ablation via the user interface of the power supply device. The ablation time and temperature for the thermal ablation are in accordance with pathological classification and the size of the tumor.

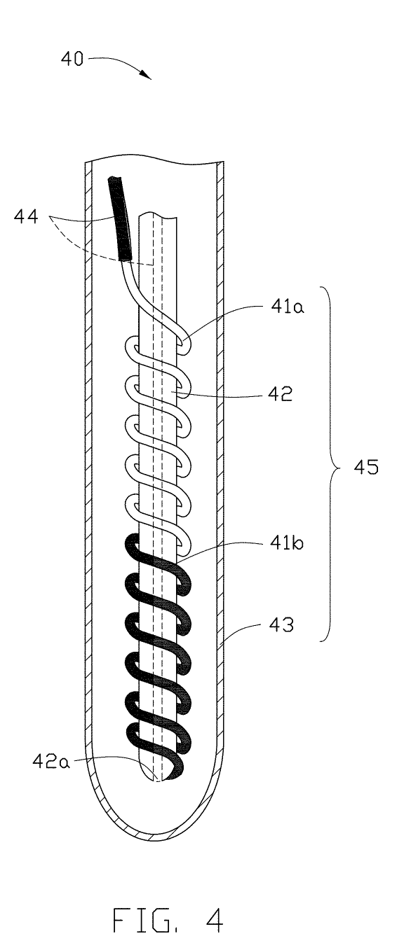

FIG. 4 is a cross-sectional side view of a heating needle 40, according to one embodiment of the present disclosure. The heating needle 40 directly contacts a target tissue and provides heat to thermally ablate the target tissue. The heating needle 40 is a rigid tubular structure, and the rigid tubular structure may include a shell in some embodiments and part or all of the shell of the heating needle 40 is resistant to physical deformation under normal atmospheric pressure and temperature (e.g., room temperature). The shell of the heating needle 40 is composed of a thermal-conductive material to efficiently conduct thermal energy to the target tissue. The thermal conductive material may be ceramic, metal, carbon fiber or other materials of high thermal conductivity. In one embodiment, the shell of the heating needle 40 is composed of stainless steel. In some embodiments, the external surface of the shell is coated with a surface treatment such as, for example nanogold, nanosilver or Teflon® to form a non-stick surface. In other embodiments, polishing or plasma treatment may be applied to the external surface of the shell to form a non-stick surface.

The heating needle 40 has a diameter of about 1.8 mm to about 3 mm and a length of about 100 mm to about 200 mm in some embodiments. Preferably, the heating needle 40 has a diameter of about 2.4 mm and a length of about 150 mm.

The heating needle 40 also includes a heating element 45 and a non-heating element 44. The heating element 45 is the part of the heating needle 40 that conducts thermal energy to the target tissue when using the tumor ablation system. The heating element 45 comprises a heating wire 41, a central tube 42 and the distal part of the shell 43.

The heating wire 41 comprises a heating wire 41a and a heating wire 41b. The heating wire 41a has a higher electrical resistance than the heating wire 41b, therefore when the heating wire 41a and the heating wire 41b are charged with electricity, the heating wire 41a and the heating wire 41b would generate different temperatures. The high resistance wire of the heating wire 41a or the heating wire 41b can be nichrome wire or tungsten wire in some embodiments, and is wrapped with insulator to prevent short circuit. The heating wire 41 is located in the heating needle 40 and is coiled on the central tube 42. The distance between each turns of the coil formed by the heating wire 41 is no greater than 5 mm. The larger the distance between each turns, the longer it takes for the heating wire 41 to be elevated to a designated temperature, and the designated temperature can be the ablation temperature. Preferably, the distance between each turns of the coil formed by the heating wire 41 is about 1 mm to about 2 mm. The coil formed by the heating wire 11 has around 5 to 20 turns in some embodiments. Preferably, the coil formed by the heating wire 41 has 8 to 10 turns.

The central tube 42 is a tube with at least one central tube orifice 42a located at the distal end of the central tube 42. The central tube orifice 42a is an entrance for the heating wire 41 to enter the central tube 42. The portion of the heating wire 41 inside the central tube 42 is electrically coupled to the non-heating element 44. The central tube orifice 42a has a diameter of about 0.3 mm to about 1 mm in some embodiments. The central tube 42 is a hollow structure centrally located in the heating needle 40, and the central tube 42 can be coiled by the heating wire 41. The central tube 42 has a diameter of about 1 mm. The length of the central tube 42 is about 120 mm to about 100 mm shorter than the heating needle 40, therefore the length of the central tube 42 is about 50 mm to about 20 mm. The central tube 42 is composed of an insulator, such as ceramic, synthetic polymer, synthetic polymer-coated ceramic and synthetic polymer-coated metal. Preferably, the central tube 42 is a ceramic tube.

When using the tumor ablation system, the heat generated by the heating wire 41 is conducted to the target tissue during thermal ablation by the distal part of the shell 43. The distal part of the shell 43 conducts the thermal energy from the heating wire 41 charged with electricity and conducts said thermal energy to the target tissue. The distal part of the shell 43 is defined by the area of the shell covering the coil formed by the heating wire 41, and the distal part of the shell 43 becomes a heated segment of the shell when the heating wire 41 is charged with electricity. The heated segment of the shell may conduct different temperatures because there are an area of the shell covering the coil formed by the heating wire 41a and another area of the shell covering the coil formed by the heating wire 41b. The distal part of the shell 43 covering the heating wire 41a will conduct different ablation temperature than the distal part of the shell 43 covering the heating wire 41b. The length of the distal part of the shell 43 corresponds to the length of the coil formed by the heating wire 41. The distal part of the shell 43 has a length of about 5 cm to about 2 cm. In one embodiment, the distal part of the shell 43 has a length of about 3 cm. When using the tumor ablation system, the user may hold the part of the heating needle 40 that is proximal to the user and distant to the distal part of the shell 43 of the heating needle 40 in order to avoid heat related injury to the user.

The non-heating element 44 is electrically coupled to the heating wire 41 and is located in the heating needle 40. The non-heating element 44 is also electrically coupled to a power supply device (not shown). In order to effectively charge the heating wire 41 with electricity, the non-heating element 44 is an insulated wire of relatively lower resistance when comparing with the heating wire 41. The non-heating element can be a copper wire, gold wire or silver wire. The non-heating element has a length of 7 cm to 15 cm.

The heating needle 40 is electrically coupled to a power supply device. The electricity provided to the heating needle 40 is DC, and the electricity has a voltage ranges from 0.1V to 10V. Preferably, the electricity provided to the heating needle 40 ranges from about 3V to about 5.5V. The ablation temperature ranges from about 70° C. to 180° C., and is positively correlated to the electric pressure of the electricity provided to the heating needle 40. The power supply device comprises an user interface, a transformer and an inverter in some embodiments. The power supply device may be electrically coupled to an AC or DC power source. If the power supply device is electrically coupled to an AC power source, then the transformer may convert the input AC into an output DC. The user interface of the power supply device controls the time and temperature of the heating needle for the thermal ablation. The surgeon may adjust the ablation time and temperature of the heating needle 40 for the thermal ablation via the user interface of the power supply device. The ablation time and temperature for the thermal ablation are in accordance with pathological classification and the size of the tumor.

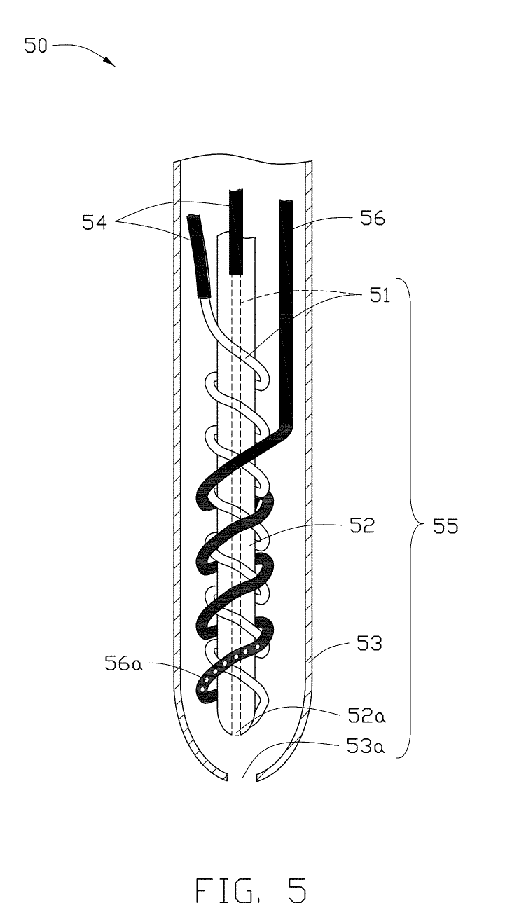

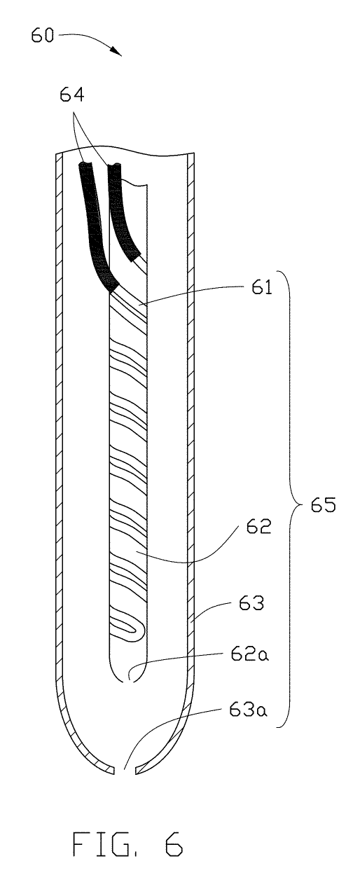

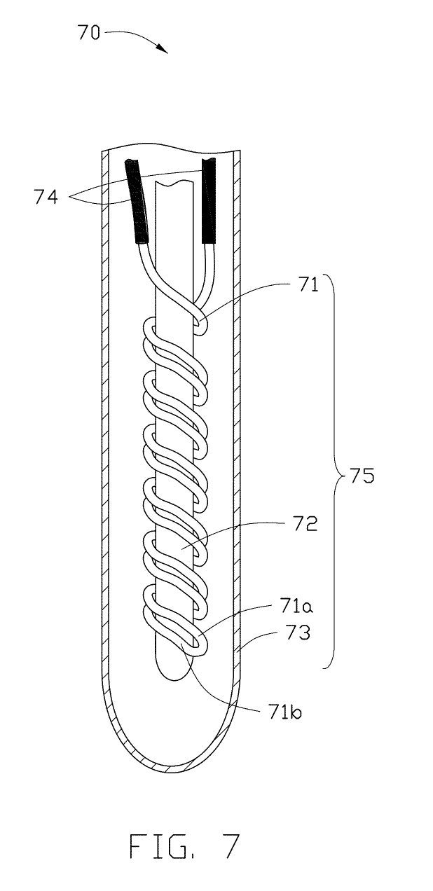

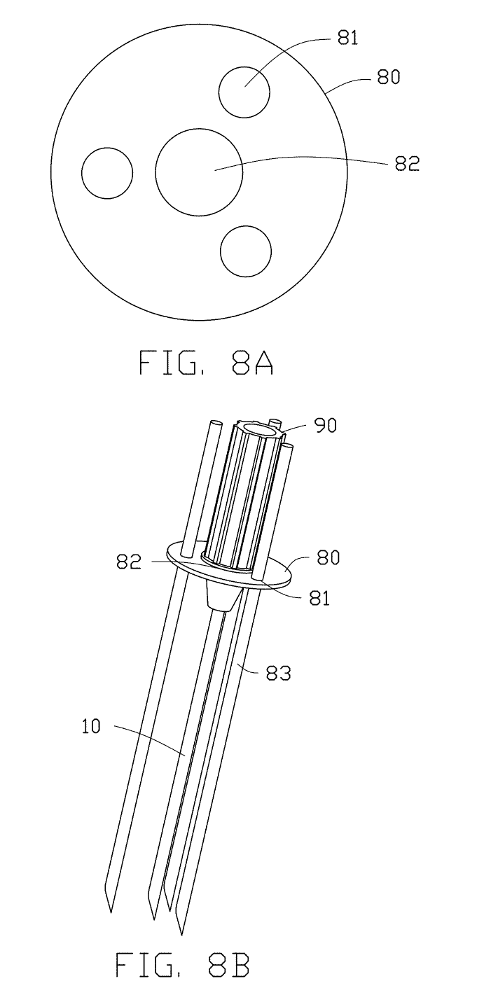

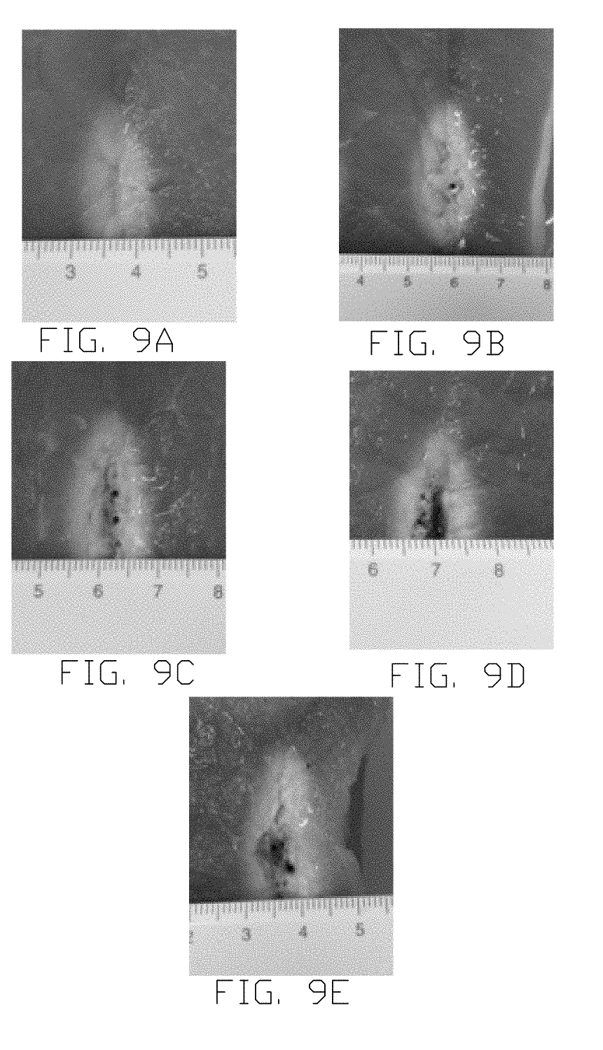

FIG. 5 is a cross-sectional side view of a heating needle 50, according to one embodiment of the present disclosure. The heating needle 50 directly contacts a target tissue, and provides heat to thermally ablate the target tissue. The heating needle 50 is a rigid tubular structure and the rigid tubular structure may include a shell in some embodiments and part or all of the shell of the heating needle 50 is resistant to physical deformation under normal atmospheric pressure and temperature (e.g. room temperature). The shell of the heating needle 50 is composed of a thermal-conductive material to efficiently conduct thermal energy to the target tissue. The thermal conductive material may be ceramic, metal, carbon fiber or other materials of high thermal conductivity. In one embodiment, the shell of the heating needle 50 is composed of stainless steel. In some embodiments, the external surface of the shell is coated with a surface treatment such as, for example nanogold, nanosilver or Teflon® to form a non-stick surface. In other embodiments, polishing or plasma treatment may be applied to the external surface of the shell to form a non-stick surface.

The heating needle 50 has a diameter of about 1.8 mm to about 3 mm and a length of about 100 mm to about 200 mm in some embodiments. Preferably, the heating needle 50 has a diameter of about 2.4 mm and a length of about 150 mm.

The heating needle 50 also includes a heating element 55 and a non-heating element 54. The heating element 55 is the part of the heating needle 50 that conducts thermal energy to the target tissue when using the tumor ablation system. The heating element 55 comprises at least one heating wire 51, a central tube 52 and the distal part of the shell 53.

The heating wire 51 is composed of high resistance wire of generating heat when charged with electricity. The high resistance wire can be nichrome wire or tungsten wire in some embodiment, and can be wrapped with insulator to prevent short circuit. The heating wire 51 is located within the heating needle 50 and is coiled on the central tube 52. The distance between each turns of the coil forming by the heating wire 51 is no greater than 5 mm in some embodiments. The larger the distance between each turns, the longer it takes for the heating wire 51 to be elevated to a designated temperature, and the designated temperature can be the ablation temperature. Preferably, the distance between each turns of the coil formed by the heating wire 51 is about 1 mm to about 2 mm. The coil formed by the heating wire 51 has around 5 to 20 turns in some embodiments. Preferably, the coil formed by the heating wire 51 has 8 to 10 turns.

The central tube 52 is a tube with at least one central tube orifice 52a located at the distal end of the central tube 52. The central tube orifice 52a is an entrance for the heating wire 51 to enter the central tube 52. The portion of the heating wire 51 inside the central tube is electrically coupled to the non-heating element 54. The central tube orifice 52a has a diameter of about 0.3 mm to about 1 mm in some embodiments. The central tube 52 is a hollow structure centrally located in the heating needle 50, and the central tube 52 can be coiled by the heating wire 51. The central tube 52 has a diameter of about 1 mm in one embodiment. The length of the central tube 52 is about 120 mm to about 100 mm shorter than the heating needle 50, therefore the length of the central tube 52 is about 50 mm to about 20 mm. The central tube 52 is composed of an insulator, such as ceramic, synthetic polymer, synthetic polymer-coated ceramic and synthetic polymer-coated metal. Preferably, the central tube 52 is a ceramic tube.

A central tube filler may be filled in the central tube 52. The central tube filler can be a thermally sensitive material, a chemical ablation agent or a medication for tumor. The thermally sensitive material can be any one of metal particles, temperature-responsive polymer particles, or polymer-coated metal particles. The chemical ablation agents can be any one of ethanol, inorganic acid solutions or inorganic base solutions. The central tube filler is removed from the central tube 52 by the central tube orifice 52a using a syringe or pump mechanism.

When using the tumor ablation system, the heat generated by the heating wire 51 is conducted to the target tissue during thermal ablation by the distal part of the shell 53. The distal part of the shell 53 conducts the thermal energy from the heating wire 51 charged with electricity and conducts said thermal energy to the target tissue. The distal part of the shell 53 is defined by the area of the shell covering the coil formed by the heating wire 51, and the distal part of the shell 53 becomes a heated segment of the shell when the heating wire 51 is charged with electricity. The length of the distal part of the shell 53 corresponds to the length of the coil formed by the heating wire 51. The distal part of the shell 53 has a length of about 5 cm to about 2 cm. In one embodiment, the distal part of the shell 53 has a length of about 3 cm. When using the tumor ablation system, the user may hold the part of the heating needle 50 that is proximal to the user and distant to the distal part of the shell 53 of the heating needle 50 in order to avoid heat related injury to the user. The distal part of the shell 53 further comprises a heating needle orifice 53a. The heating needle orifice 53a is located at the distal end of the distal part of the shell 53. The heating needle orifice 53a has a diameter of about 0.5 mm to about 1 mm. When the central tube filler reaches the space between the heating wire 51 and the shell of the heating needle 50, the central tube filler can be removed from the heating needle 50 by the heating needle orifice 53a. The central tube filler can be removed from the heating needle 50 before, during or after the thermal ablation.

A peripheral passage 56 is located in the heating needle 50. The peripheral passage is a tubular structure that spirally surrounds the central tube 52 in one embodiment. The peripheral passage further comprises a peripheral passage filler. The peripheral passage filler can be an inert gas, a thermally sensitive material, a chemical ablation agent, a cryoablation agent or a medication for tumor. The inert gas can be any one of helium (He), neon (Ne), argon (Ar), krypton (Kr) or xenon (Xe). The thermally sensitive material can be any one of metal particles, temperature-responsive polymer particles, or polymer-coated metal particles. The chemical ablation agents can be any one of ethanol, inorganic acid solutions or inorganic base solutions. The cryoablation agent can be liquid nitrogen (N₂) or high-pressure argon (Ar). The peripheral passage filler can be removed from the heating needle 50 by the peripheral passage orifice 56a using a syringe or pump mechanism.

The non-heating element 54 is electrically coupled to the heating wire 51 is located in the heating needle 50. The non-heating element 54 is also electrically coupled to a power supply device (not shown). In order to effectively charge the heating wire 51 with electricity, the non-heating element 54 is an insulated wire of relatively lower resistance compared with the heating wire 51. The non-heating element can be a conductor such as a copper wire, gold wire or silver wire and has a length of about 7 cm to about 15 cm.

The heating needle 50 is electrically coupled to a power supply device. The electricity provided to the heating needle 50 is direct current (DC), and the electricity has a voltage ranging from about 0.1V to about 10V. Preferably, the electricity provided to the heating needle 50 ranges from about 3V to about 5.5V. The ablation temperature ranges from about 70° C. to 180° C., and is positively correlated to the electric pressure of the electricity provided to the heating needle 50. The power supply device comprises an user interface, a transformer and an inverter in some embodiments. The power supply device may be electrically coupled to an AC or DC power source. If the power supply device is electrically coupled to an AC power source, then the transformer may convert the input AC into an output DC. The user interface of the power supply device controls the time and temperature of the heating needle for the thermal ablation. The surgeon may adjust the ablation time and temperature of the heating needle 50 for the thermal ablation via the user interface of the power supply device. The ablation time and temperature for the thermal ablation are in accordance with pathological classification and the size of the tumor.

FIG. 6 is a cross-sectional side view of a heating needle 60, according to one embodiment of the present disclosure. The heating needle 60 directly contacts a target tissue and provides heat to thermally ablate the target tissue. The heating needle 60 is a rigid tubular structure and the rigid tubular structure may include a shell in some embodiments and part or all of the shell of the heating needle 60 is resistant to physical deformation under normal atmospheric pressure and temperature (e.g., room temperature). The shell of the heating needle 60 is composed of a thermal-conductive material to efficiently conduct thermal energy to the target tissue. The thermal conductive material may be ceramic, metal, carbon fiber or other materials of high thermal conductivity. In one embodiment, the shell of the heating needle 60 is composed of stainless steel. In some embodiments, the external surface of the shell is coated with a surface treatment such as, for example nanogold, nanosilver or Teflon® to form a non-stick surface. In other embodiment, polishing or plasma treatment may be applied to the external surface of the shell to form a non-stick surface.

The heating needle 60 has a diameter of about 1.8 mm to about 3 mm and a length of about 100 mm to about 200 mm in some embodiments. Preferably, the heating needle 60 has a diameter of about 2.4 mm and a length of about 150 mm.

The heating needle 60 also includes a heating element 65 and a non-heating element 64. The heating element 65 is the part of the heating needle 60 that conducts thermal energy to the target tissue when using the tumor ablation system. The heating element 65 comprises at least one heating layer 61, a central tube 62 and the distal part of the shell 63.

The heating layer 61 is composed of high resistance material capable of generating heat when charged with electricity. The high resistance material can be nichrome or tungsten in some embodiments. The heating layer 61 is located within the heating needle 60 and is electroplated on the central tube 62. The electroplating pattern of the heating layer 61 is spirally surrounding the central tube 62. The distance between each turns of the heating layer 61 is no greater than 5 mm in some embodiments. The larger the distance between each turns, the more time it requires for the heating layer 61 to be elevated to a designated temperature, and the designated temperature can be the ablation temperature. Preferably, the distance between each turns of the heating layer 61 is about 1 mm to about 2 mm. The heating layer 61 is turned on the distal end of the central tube 62, and the turned part of the heating layer 61 is electrically coupled to the non-heating element 64 on the proximal end of the central tube 62. The heating layer 61 has around 5 to 20 turns on the central tube 62 in some embodiments. Preferably, the heating layer 61 has 8 to 10 turns.

The non-heating element 64 is electrically coupled to the heating layer 61 and is located in the heating needle 60. The non-heating element 64 is also electrically coupled to a power supply device (not shown). In order to effectively charge the heating layer 61 with electricity, the non-heating element 64 is an insulated wire of relatively lower resistance compared to the heating layer 61. The non-heating element 64 can be a conductor such as a copper wire, gold wire or silver wire and has a length of about 7 cm to about 15 cm.

The central tube 62 is a tube with at least one central tube orifice 62a located at the distal end of the central tube 62. The central tube 62 has a diameter of 1 mm. The length of the central tube 62 is 120 mm to 100 mm shorter than the heating needle 60, therefore the length of the central tube 62 is about 50 mm to 20 mm. The central tube 62 is composed of insulator, such as ceramic, synthetic polymer, synthetic polymer-coated ceramic and synthetic polymer-coated metal. Preferably, the central tube 62 is a ceramic tube.

A central tube filler may be filled in the central tube 62. The central tube filler can be a thermally sensitive material, a chemical ablation agent or a medication for tumor. The thermally sensitive material can be any one of metal particles, temperature-responsive polymer particles, or polymer-coated metal particles. The chemical ablation agents can be any one of ethanol, inorganic acid solutions or inorganic base solutions. The central tube filler is removed from the central tube 62 by the central tube orifice 62a using a syringe or pump mechanism.

When using the tumor ablation system, the heat generated by the heating layer 61 is conducted to the target tissue during thermal ablation by the distal part of the shell 63. The distal part of the shell 63 conducts the thermal energy from the heating layer 61 with electricity and conducts said thermal energy to the target tissue. The distal part of the shell 63 is defined by the area of the shell covering the turns of the heating layer 61 on the central tube 62, and the distal part of the shell 63 becomes a heated segment when the heating layer 61 is charged with electricity. The length of the distal part of the shell 63 corresponds to the length of the heating layer 61 on the central tube 62. The distal part of the shell 63 has a length of about 5 cm to about 2 cm. In one embodiment, the distal part of the shell 63 has a length of about 3 cm.

When using the tumor ablation system, the user may hold the part of the heating needle 60 that is proximal to the user and distant to the distal part of the shell 63 of the heating needle 60 in order to avoid heat related injury to the user. The distal part of the shell 63 further comprises a heating needle orifice 63a. The heating needle orifice 63a is located at the distal end of the distal part of the shell 63. The heating needle orifice 63a has a diameter of about 0.5 mm to 1 mm. When the central tube filler reaches the space between the heating layer 61 and the shell of the heating needle 60, the central tube filler can be removed from the heating needle 60 by the heating needle orifice 63a. The central tube filler can be removed from the heating needle 60 before, during or after the thermal ablation.

FIG. 7 is a cross-sectional side view of a heating needle 70, according to one embodiment of the present disclosure. The heating needle 70 directly contacts a target tissue and provides heat to thermally ablate the target tissue. The heating needle 70 is a rigid tubular structure and the rigid tubular structure may include a shell in some embodiments and part or all of the shell of the heating needle 70 is resistant to physical deformation under normal atmospheric pressure and temperature (e.g., room temperature). The shell of the heating needle 70 is composed of a thermal-conductive material to efficiently conduct thermal energy to the target tissue. The thermal conductive material may be ceramic, metal, carbon fiber or other materials of high thermal conductivity. In one embodiment, the shell of the heating needle 70 is composed of stainless steel. In some embodiments, the external surface of the shell is coated with nanogold, nanosilver or Teflon® to form a non-stick surface. In other embodiments, polishing or plasma treatment may be applied to the external surface of the shell to form a non-stick surface.

The heating needle 70 has a diameter of about 1.8 mm to about 3 mm and a length of 100 mm to 200 mm Preferably, the heating needle 10 has a diameter of about 2.4 mm and a length of 150 mm.

The heating needle 70 also includes a heating element 75 and a non-heating element 74. The heating element 75 is the part of the heating needle 70 that conducts thermal energy to the target tissue when using the tumor ablation system. The heating element 75 comprises at least one heating wire 71, a central cylindrical object 72 and the distal part of the shell 73.

The heating wire 71 is composed of high resistance wire capable of generating heat when charged with electricity. The high resistance wire can be nichrome wire or tungsten wire in some embodiments, and can be wrapped with insulator to prevent short circuit. The heating wire 71 is located within the heating needle 70 and is coiled on the central cylindrical object 72. The heating wire 71 is folded on the distal end of the central cylindrical object 72, and the heating wire 71 comprises an outward strand 71a and an inward strand 71b. The outward strand 71a is turned on the distal end of the central cylindrical object 72 to form the inward strand 71b. The inward strand 71b is folded back along the outward strand 71a. The outward strand 71a and the inward strand 71b are jointly coiled on the central cylindrical object 72. The distance between each turns of the coil formed by the heating wire 71 is no greater than 5 mm. The larger the distance between each turns, the more time it requires for the heating wire 71 to be elevated to a designated temperature, and the designated temperature can be the ablation temperature. Preferably, the distance between each turns of the coil formed by the heating wire 71 is about 1 mm to about 2 mm. The coil formed by the heating wire 71 has around 5 to 20 turns. In some embodiments, the coil formed by the heating wire 71 has 8 to 10 turns.

The central cylindrical object 72 is centrally located in the heating needle 70. The central cylindrical object 72 can be a tube or a cylinder, and the central cylindrical object 72 can be coiled by the heating wire 71. The central cylindrical object 72 has a diameter of about 1 mm. The length of the central cylindrical object 72 is about 120 mm to about 100 mm shorter than the heating needle 70, therefore the length of the central cylindrical object 72 is about 50 mm to about 20 mm. The central cylindrical object 72 is composed of insulator, such as ceramic, synthetic polymer, synthetic polymer-coated ceramic and synthetic polymer-coated metal. Preferably, the central cylindrical object 72 is a ceramic cylinder.

When using the tumor ablation system, the heat generated by the heating wire 71 is conducted to the target tissue during thermal ablation by the distal part of the shell 73. The distal part of the shell 73 conducts the thermal energy from the heating wire 71 charged with electricity and conducts said thermal energy to the target tissue. The distal part of the shell 73 is defined by the area of the shell covering the coil formed by the heating wire 71, and the distal part of the shell 73 becomes a heated segment of the shell when the heating wire 71 is charged with electricity. The length of the distal part of the shell 73 corresponds to the length of the coil formed by the heating wire 71. The distal part of the shell 73 has a length of about 5 cm to about 2 cm. In one embodiment, the distal part of the shell 73 has a length of about 3 cm. When using the tumor ablation system, the user may hold the part of the heating needle 70 that is proximal to the user and distant to the distal part of the shell 73 of the heating needle 70 in order to avoid heat related injury to the user.

The non-heating element 74 is electrically coupled to the heating wire 71 and located in the heating needle 70. The non-heating element 74 is located in the space between the central cylindrical object 72 and the distal part of the shell 73. The non-heating element 74 is also electrically coupled to a power supply device. In order to effectively charge the heating wire 71 with electricity, the non-heating element 74 is an insulated wire of relatively lower resistance compared to the heating wire 71. The non-heating element can be a conductor such as a copper wire, gold wire or silver wire and has a length of about 7 cm to about 15 cm.

The heating needle 70 is electrically coupled to a power supply device. The electricity provided to the heating needle 70 is direct current (DC), and the electricity has a voltage ranging from about 0.1V to about 10V. Preferably, the electricity provided to the heating needle 70 ranges from about 3V to about 5.5V. The ablation temperature ranges from about 70° C. to about 180° C., and is positively correlated to the electric pressure of the electricity provided to the heating needle 70. The power supply device comprises an user interface, a transformer and an inverter in some embodiments. The power supply device may be electrically coupled to an AC or DC power source. If the power supply device is electrically coupled to an AC power source, then the transformer may convert the input AC into an output DC. The user interface of the power supply device controls the time and temperature of the heating needle for the thermal ablation. The surgeon may adjust the ablation time and temperature of the heating needle 70 for the thermal ablation via the user interface of the power supply device. The ablation time and temperature for the thermal ablation are in accordance with pathological classification and the size of the tumor.

Alternatively, a connecting device is electrically coupled to the power supply device. The connecting device can be electrically coupled to the heating needle 10, the heating needle 20, the heating needle 30, the heating needle 40, the heating needle 50, the heating needle 60 or the heating needle 70 when being electrically coupled to the power supply device. Preferably, the connecting device is electrically coupled to the non-heating element 14 of the heating needle 10, the non-heating element 24 of the heating needle 20, the non-heating element 34 of the heating needle 30, the non-heating element 44 of the heating needle 40, the non-heating element 54 of the heating needle 50, the non-heating element 64 of the heating needle 60 or the non-heating element 74 of the heating needle 70. The connecting device has one or more wires wrapped with insulators, such as fabrics or polymers. With the connecting device, the user may experience more maneuverability when using the heating needle 10, the heating needle 20, the heating needle 30, the heating needle 40, the heating needle 50, the heating needle 60 or the heating needle 70. The length of the connecting device ranges from 50 cm to 3 m. Preferably, the connecting device has a length of 1 m.

Before conducting the thermal ablation by using the heating needle 10, the heating needle 20, the heating needle 30, the heating needle 40, the hating needle 50, the heating needle 60 or the heating needle 70, the surgeon would need to identify the location of tumor by histology analysis, by visual examination or by medical imaging instruments. The medical imaging instrument used to identify the location of the tumor may include, but is not limited to: ultrasound imaging, X-ray, computed tomography (CT) or magnetic resonance imaging (MRI). The medical imaging instrument can be used to assist the process of the thermal ablation. If the tumor is located in a lumen, a subdermal position or a subcutaneous position, an ultrasound imaging instrument may be used in the process of the thermal ablation to demonstrate the position of the tumor. The surgeon may configure the ablation time and the temperature on the user interface of the power supply device. The surgeon may then place or insert one or more of the heating needle 10, the heating needle 20, the heating needle 30, the heating needle 40, the hating needle 50, the heating needle 60 or the heating needle 70 on the target tissue or in the target tissue. The ultrasound imaging instrument may be used for assisting the surgeon to position any of the above heating needles. If more than one of the heating needle 10, the heating needle 20, the heating needle 30, the heating needle 40, the heating needle 50, the heating needle 60 or the heating needle 70 is inserted or placed, then the distance between each of the inserted or placed heating needle should be about 1.5 cm to 2 cm. After a sufficient amount of time, the surgeon may remove the inserted or placed heating needle from the target tissue. When removing the heating needle 10, the heating needle 20, the heating needle 30, the heating needle 40, the hating needle 50, the heating needle 60 or the heating needle 70, any of the above heating needle used may be adjusted to a temperature that is 10° C. to 30° C. lower than the ablation temperature to prevent unnecessary tissue damage. The temperature adjustment during the removal of the heating needle 10, the heating needle 20, the heating needle 30, the heating needle 40, the hating needle 50, the heating needle 60 or the heating needle 70 also destroys the tumor cells attached on any of the above heating needle used to prevent cancer metastasis.

After the thermal ablation by using the heating needle 10, the heating needle 20, the heating needle 30, the heating needle 40, the hating needle 50, the heating needle 60 or the heating needle 70, the surgeon may surgically remove the tumor. The ablated target tissue can also be exposed to therapeutic radiation, or be chemically ablated.

According to FIG. 1, FIG. 2, FIG. 3, FIG. 4, FIG. 5, FIG. 6 and FIG. 7, a heating needle filler can be filled in the space between the heating wire 11 and the shell of the heating needle 10, the space between the heating wire 21 and the shell of the heating needle 20, the space between the heating wire 31 and the shell of the heating needle 30, the space between the heating wire 41 and the shell of the heating needle 40, the space between the heating wire 51 and the shell of the heating needle 50, the space between the heating layer 61 and the heating needle 60 or the space between the heating wire 71 and the heating needle 70. The heating needle filler can be an inert gas, an ultrasound contrast agent or a thermally sensitive material. The inert gas can be any one of helium (He), neon (Ne), argon (Ar), krypton (Kr) or xenon (Xe). The ultrasound contrast agent can be any commercially approved microbubble-based contrast agents, such as Optson™, Definity®, SonoVue® or Sonazoid™. The thermally sensitive material can be any one of metal particles, temperature-responsive polymer particles, or polymer-coated metal particles.

The heating needle filler in the heating needle 20 can be removed by the heating needle orifice 23a; the heating needle filler in the heating needle 50 can be removed by the heating needle orifice 53a; the heating needle filler in the heating needle 60 can be removed by the heating needle 63a. The above heating needle filler can be removed from the heating needle 20, the heating needle 50 and the heating needle 60 using a syringe or pump mechanism. The heating needle filler removed from the heating needle 20, the heating needle 50 and the heating needle 60 can be a chemical ablation agent or a medication for tumor. The chemical ablation agents can be any one of ethanol, inorganic acid solutions or inorganic base solutions. The heating needle filler can be removed from the heating needle 20, the heating needle 50 and the heating needle 60 before, during or after the thermal ablation.

The heating needle 20, the heating needle 50 and the heating needle 60 may be filled with both the heating needle filler and the central tube filler. The heating needle 20, the heating needle 50 and the heating needle 60 may be filled only with the heating needle filler, or filled only with the central tube filler. The central tube filler can be removed from the heating needle 20, the heating needle 50 and the heating needle 60 before, during or after the thermal ablation.

FIG. 8A and FIG. 8B illustrate a holder in accordance with one embodiment of the present disclosure. The holder 80 is a flat structure supporting and fixing one or more components of the tumor ablation system. The holder 80 includes at least one major aperture 82 and 3 minor aperture 81 in some embodiments. The distance between the major aperture 82 and the minor aperture 81 can be from about 50 mm to about 80 mm. The major aperture 82 has a diameter of 80 mm to 120 mm for supporting and fixing the connecting device 90. The connecting device 90 is a polymer-wrapped wire coupled to the heating needle 10. The minor aperture 81 has a diameter of 40 mm to 70 mm for supporting and fixing the measuring needle 83 or other components of the tumor ablation system. The measuring needle 83 is used to measure the temperature of surrounding environment when the thermal therapeutic energy is generated. The measuring needle 83 may be used to measure the temperature of the target tissue when the thermal ablation is taken place, and multiple measuring needle 83 may be used to measure temperatures on different locations of the target tissue. The measuring needle 83 can be a thermocoupler or a thermistor. The relative position between the measuring needle 83 and the connecting device 90 are fixed by the holder 80.

Additionally, a syringe can be fixed and supported by the holder 80. The syringe can be filled with an inert gas, an ultrasound contrast agent, a thermally sensitive material, a chemical ablation agent or a medication for tumor. The inert gas can be any one of helium (He), neon (Ne), argon (Ar), krypton (Kr) or xenon (Xe). The ultrasound contrast agent can be any commercially approved microbubble-based contrast agents, such as Optson™, Definity®, SonoVue® or Sonazoid™. The thermally sensitive material can be any one of metal particles, temperature-responsive polymer particles, or polymer-coated metal particles. The chemical ablation agents can be any one of ethanol, inorganic acid solutions or inorganic base solutions. The inert gas, the ultrasound contrast agent, the thermally sensitive material or the therapeutic agent are injected to the target tissue before, during or after the thermal ablation.

The present disclosure will now be described more specifically with reference to the following examples, which are provided for the purpose of demonstration rather than limitation.

### EXAMPLES

### Materials and Methods

Tumor ablation system. The tumor ablation system comprises a heating needle, a connecting device, a power supply device and a measuring needle. The heating needle is the heating needle 10, and the length of the heating element is 2 cm. The measuring needle is a thermocoupler.

In-vitro muscle tissue. The in-vitro muscle tissue is obtained from skeletal muscle of the pig. The in-vitro muscle tissue has a weight of 30 g.

In-vivo target tissue. The target tissue is a canine melanoma located in the nasal cavity of a dog.

### Example 1: The Ablation Area, Temperature and Output Voltage

The power supply device is adjusted to output electricity of 3.0 V, 4.0 V, 4.5 V, 5.0 V and 5.5 V to the heating needle. The heating needle then contacts the in-vitro muscle tissue to conduct thermal ablation. The ablation time is 5 minutes. During the thermal ablation, the temperature of the in-vitro muscle tissue is measured by the measuring needle. The diameter of the ablation area is measured after the thermal ablation. Table 1 demonstrates the temperature and the ablation area of the in-vitro muscle tissue when ablated by the heating needle of different output voltages.

TABLE 1
The temperatures and ablation area with the heating needle of
different voltages.

| | Temperature of the | Diameter of ablation area |
|---|---|---|
| Output Voltage | in-vitro muscle tissue (° C.) | (cm) in 5 minutes |
| 3.0 V | 80 ± 5° C. | 1.2 ± 0.1 |
| 4.0 V | 90 ± 5° C. | 1.3 ± 0.1 |
| 4.5 V | 100 ± 5° C. | 1.4 ± 0.1 |
| 5.0 V | 120 ± 5° C. | 1.5 ± 0.1 |
| 5.5 V | 150 ± 5° C. | 1.5 ± 0.1 |

Referring to Table 1, the ablation area is larger when higher output voltage is provided to the heating needle, therefore the ranges of ablation are positively correlated to the output voltage from 3.0 V to 5.0 V. The temperature of the in-vitro muscle tissue during ablation is higher when higher output voltage is provided to the heating needle, therefore the temperatures of the in-vitro muscle tissues are positively correlated to the output voltage from 3.0 V to 5.5 V.

FIG. 9A is the appearance of the in-vitro muscle tissue when ablated by the heating needle with a 3.0 V output voltage. FIG. 9B is the appearance of the in-vitro muscle tissue when ablated by the heating needle with a 4.0 V output voltage. FIG. 9C is the appearance of the in-vitro muscle tissue when ablated by the heating needle with a 4.5 V output voltage. FIG. 9D is the appearance of the in-vitro muscle tissue when ablated by the heating needle with a 5.0 V output voltage. FIG. 9E is the appearance of the in-vitro muscle tissue when ablated by the heating needle of 5.5 V output voltage. The ablation area in FIG. 9E and FIG. 9D are significantly larger than the ablation area in FIG. 9C, FIG. 9B and FIG. 9A.

### Example 2: The Ablation Time and the Ablation Area with the Heating Needle of a Designated Voltage

The power supply device is adjusted to output electricity of 4.5 V to the heating needle. The heating needle then contacts the in-vitro muscle tissue to conduct thermal ablation. During the thermal ablation, the diameter of the ablation area is measured. The experiment is triplicated. Table 2 demonstrates the time required for the ablation area to reach certain diameters.

TABLE 2
The ablation time and the ablation area with the heating needle of
a designated voltage.

| | | | | Average time |
|---|---|---|---|---|
| | | | | required to |
| | Time required for the ablation area | | | reach # cm of |
| | to reach # mm in diameter (sec) | | | the diameter of |
| Diameter of | 1^{st} | 2^{nd} | 3^{rd} ex- | ablation area |
| ablation area | experiment | experiment | periment | (sec) |
| 1.0 mm | 15 | 13 | 15 | 14.3 |
| 2.0 mm | 30 | 26 | 30 | 29.7 |
| 3.0 mm | 70 | 65 | 75 | 70.0 |
| 4.0 mm | 120 | 110 | 105 | 111.7 |
| 5.0 mm | 200 | 180 | 165 | 181.7 |

Referring to Table 2, longer the ablation time results in larger ablation area. The ablation time required for the ablation area to reach certain diameters are similar in the 1^{st}, the 2^{nd} and the 3^{rd} experiment. The ablation area is positively correlated to the ablation time.

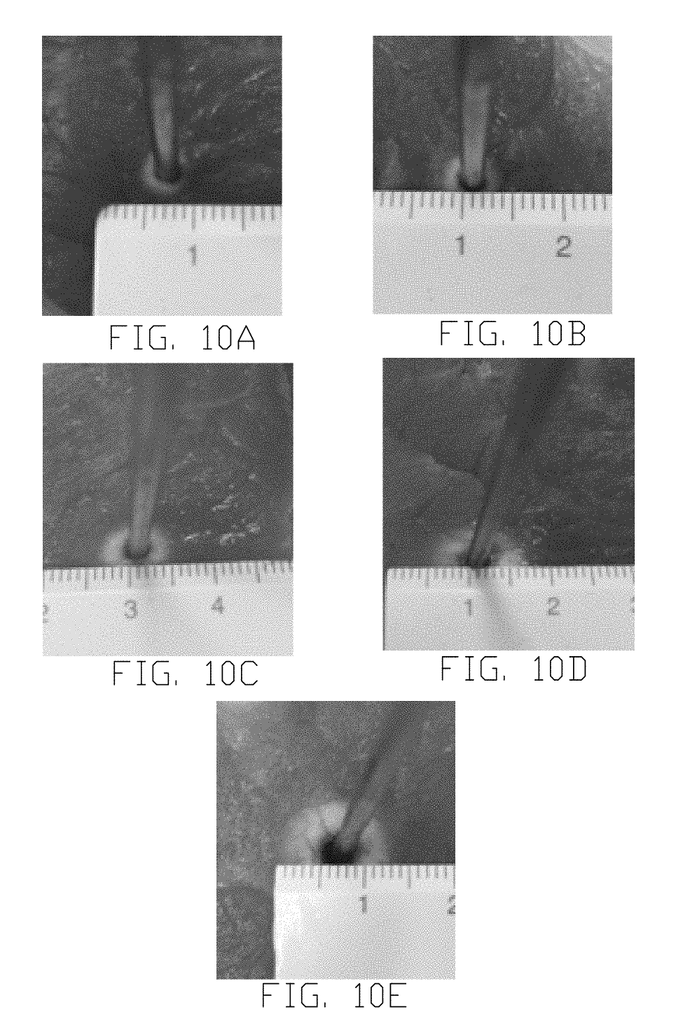

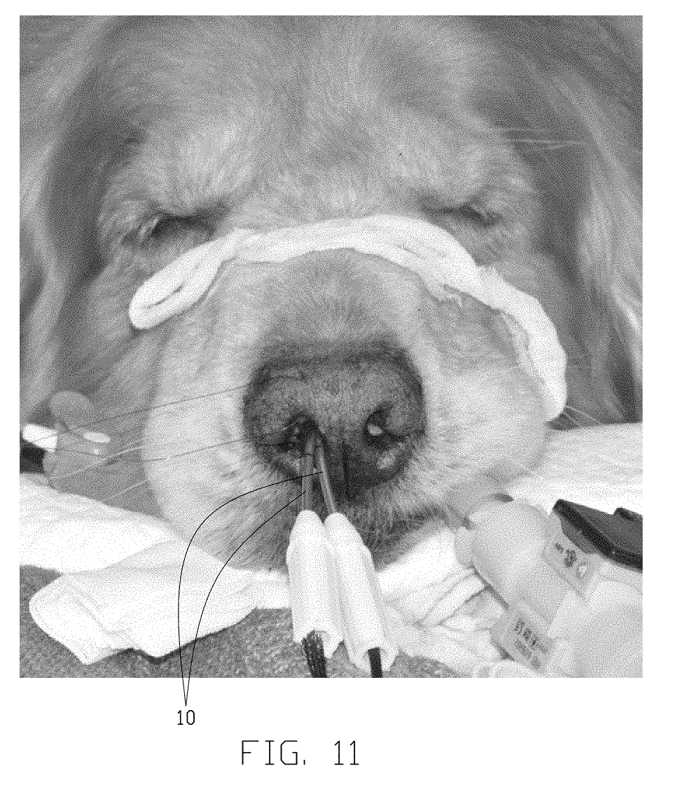

FIG. 10A is the appearance of the in-vitro muscle tissue when ablation area reaches 1.0 mm in diameter. FIG. 10B is the appearance of the in-vitro muscle tissue when ablation area reaches 2.0 mm in diameter. FIG. 10C is the appearance of the in-vitro muscle tissue when ablation area reaches 3.0 mm in diameter. FIG. 10D is the appearance of the in-vitro muscle tissue when ablation area reaches 4.0 mm in diameter. FIG. 10E is the appearance of the in-vitro muscle tissue when ablation area reaches 5.0 mm. Table 2, FIG. 10A, FIG. 10B FIG. 10C, FIG. 10D and FIG. 10E demonstrate the thermal ablation conducted by the tumor ablation system of the present disclosure may rapidly elevate the temperature to form an ablation area sufficiently large on the target tissue.

### Example 3: Therapeutic Effects of the Tumor Ablation System

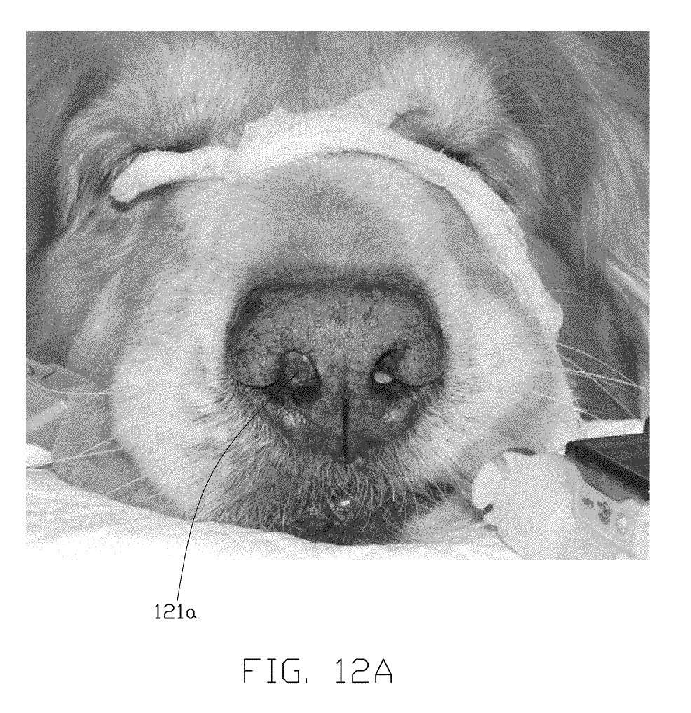

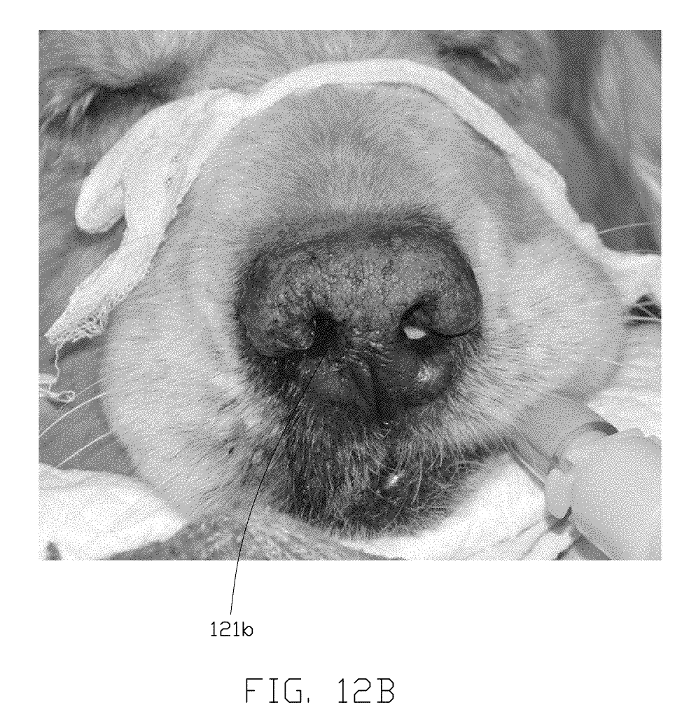

Referring to FIG. 11, FIG. 12A and FIG. 12B, a dog with melanoma in the nasal cavity is anesthetized. The melanoma in the nasal cavity is identified as the target tissue of the thermal ablation. Two heating needle 10 are inserted and positioned in the nasal cavity by using CT. The thermal ablation is conducted by the ablation temperature of 100° C. and the ablation time of 300 seconds.

FIG. 11 is the appearance of the surgery. Two heating needle 10 are inserted in the nasal cavity of the dog. The heating needle 10 are charged with electricity when conducting the thermal ablation. The distal ends of each of the heating needle 10 are in contact with the melanoma. FIG. 12A is the appearance of the melanoma 121a before the surgery. FIG. 12B is the appearance of the melanoma 121b after the surgery. During the thermal ablation conducted by the tumor ablation system, the hemorrhage from the target tissue is contained. The therapeutic effects of the tumor ablation system can be seen from FIG. 12A and FIG. 12B. The melanoma 121a is significantly visible when comparing to the melanoma 121b. The melanoma 121b is necrotized after the thermal ablation.

The embodiments shown and described above are only examples. Many details are often found in the art such as the other features of a circuit board assembly. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including the full extent established by the broad general meaning of the terms used in the claims. It will therefore be appreciated that the embodiments described above may be modified within the scope of the claims.

1. A tumor ablation system, comprising:
a heating needle having a rigid tubular shell, the heating needle comprising a heating element and a non-heating element, the heating element having at least one heating wire, a central cylindrical object and a distal part of the shell, wherein the heating wire and the central cylindrical object are inside the shell, the heating wire is electrically coupled to the non-heating element and coiled on the central cylindrical object, the distal part of the shell covers the coil formed by the heating wire on the central cylindrical object, and when the heating wire is charged with electricity the heating wire generates thermal energy that is conducted to the distal part of the shell; and
a power supply device coupled to the non-heating element, the power supply device providing electricity to the heating needle.

2. The tumor ablation system of claim 1, further comprising a connecting device, the connecting device being electrically coupled to the power supply device and the non-heating element.

3. The tumor ablation system of claim 1, further comprising a measuring needle for measuring the temperature of a surrounding environment when the thermal energy is generated.

4. The tumor ablation system of claim 1, further comprising a holder supporting one or more heating needles and measuring needles.

5. The tumor ablation system of claim 1, further comprising a peripheral passage, the peripheral passage spirally surrounding the heating wire.

6. The tumor ablation system of claim 5, wherein the peripheral passage comprises a distal end and a proximal end, and the distal end has a plurality of orifices.

7. The tumor ablation system of claim 6, wherein the peripheral passage further comprises a peripheral passage filler, and the peripheral passage filler is filled in the peripheral passage and removed from the peripheral passage by the plurality of orifices of the peripheral passage.

8. The tumor ablation system of claim 7, wherein the peripheral passage filler is an inert gas, a thermally sensitive material, a chemical ablation agent, a cryoablation agent or a medication for tumor.

9. A heating needle having a rigid tubular shell, comprising:
a heating element and a non-heating element, the heating element having at least one heating wire, a central cylindrical object and a distal part of the shell, wherein the heating wire and the central cylindrical object are inside the shell, the heating wire is electrically coupled to the non-heating element and coiled on the central cylindrical object, the distal part of the shell covers the coil formed by the heating wire on the central cylindrical object, and when the heating wire is charged with electricity the heating wire generates thermal energy that is conducted to the distal part of the shell.

10. The heating needle of claim 9, further comprising a heating needle filler, wherein the heating needle filler is filled in the space between the shell of the heating needle and the central cylindrical object.

11. The heating needle of claim 10, wherein the shell of the heating needle has a distal end and a proximal end, a heating needle orifice is located at the distal end of the shell of the heating needle, and the heating needle filler can be removed by the heating needle orifice from the space between the shell of the heating needle and the central cylindrical object.

12. The heating needle of claim 10, wherein the heating needle filler is an inert gas, a thermally sensitive material or an ultrasound contrast agent.

13. The heating needle of claim 11, wherein the heating needle filler is a chemical ablation agent or a medication for tumor.

14. The heating needle of claim 9, wherein the central cylindrical object is a central tube.

15. The heating needle of claim 14, wherein the central tube comprises a central tube filler, and the central tube filler is filled in the central tube.

16. The heating needle of claim 15, wherein the central tube has a distal end and a proximal end, and a central tube orifice is located at the distal end of the central tube.

17. The heating needle of claim 16, wherein the central tube filler can be removed from the central tube by way of the central tube orifice.

18. The heating needle of claim 17, wherein the central tube filler is a chemical ablation agent, a thermally sensitive material or a medication for tumor.

19. The heating needle of claim 14, wherein the central tube is composed of an insulator, the insulator can be ceramic, synthetic polymer, synthetic polymer-coated ceramic, or synthetic polymer-coated metal.

20. The heating needle of claim 9, wherein the heating wire can be a nichrome wire or a tungsten wire.

21. The heating needle of claim 9, wherein the shell of the heating needle can be ceramic, metal or carbon fiber.

22. The heating needle of claim 9, wherein the heating wire and the non-heating element form more than one electrical connection, and one or more electrical connection is selected to generate thermal energy, and one or more segments of the distal part of the shell is heated.

23. The heating needle of claim 9, wherein the heating wire comprises of at least two materials of different electrical resistance.

24. A method for treatment of tumor, comprising steps of:
a) identifying a location of the tumor; and
b) thermally ablating the tumor by using a tumor ablation system, the tumor ablation system comprising a heating needle having a rigid tubular shell, the heating needle comprising a heating element and a non-heating element, the heating element having at least one heating wire, a central cylindrical object and a distal part of the shell, wherein the heating wire and the central cylindrical object are inside the shell, the heating wire is electrically coupled to the non-heating element and coiled on the central cylindrical object, wherein the distal part of the shell covers the coil formed by the heating wire on the central cylindrical object, and when the heating wire is charged with electricity the heating wire generates thermal energy that is conducted to the distal part of the shell; and a power supply device coupled to the non-heating element, the power supply device providing electricity to the heating needle.

25. The method of claim 24, wherein the electricity provided by the power supply device to the heating needle has an electric pressure, and the electric pressure is ranged from 0.1V to 10V.

26. The method of claim 24, wherein the time of thermally ablating the tumor by thermal energy ranges from 1 second to 600 seconds.

27. The method of claim 24, wherein the temperature of the distal part of the shell when conducting thermal energy ranges from about 30° C. to 300° C.

28. The method of claim 24, wherein the method further comprises surgically removing the tumor after thermally ablating the tumor.

29. The method of claim 24, wherein the method further comprises exposing the tumor to therapeutic radiation after thermally ablating the tumor.

30. The method of claim 24, further comprising a step of: exposing the tumor to one or more chemical ablation agent after thermally ablating the tumor.

31. The method of claim 24, further comprising a step of: exposing the tumor to one or more cryoablation agent after thermally ablating the tumor.
